(19) United States
(12) Patent Application Publication
(10) Pub. No.: US 2008/0308098 A1
(43) Pub. Date: Dec. 18, 2008

(54) Endotracheal intubation device

(75) Inventors: John Schwartz, Williamston, MI (US); Richard Schwartz, Evans, GA (US)

Correspondence Address: Ian C. McLeod;IAN C. MCLEOD, P.C., 2190 Commons Parkway, Okemos, MI, 48864, US

(73) Assignee: AI Medical Devices, Williamston, MI (US)

(21) Appl. No.: 11/820,117

(22) Filed: Jun. 18, 2007

(51) Int. Cl. A61M 16/04

(52) U.S. Cl. 128/200.26

(57) ABSTRACT

An endotracheal intubation device, optionally having a curvable portion and internal optics or a viewing device is provided. The curvable portion of a tubular element, over which is placed an endotracheal tube, curves in a controlled manner to a curved conformation from a straight or less curved configuration so as to facilitate the insertion of the endotracheal tube into a patient. A length of a proximal end of the tubular element projects over a grip portion of the device, which makes the device easier to use than other devices used for airway intubation.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

(1) Field of the Invention

The present invention relates generally to endotracheal intubation devices, and more specifically to a endotracheal devices having a curvable portion.

(2) Description of the Related Art

U.S. Pat. No. 2,975,785 to Sheldon discloses an optical viewing instrument comprising an endoscope sheath and a plurality of tube elements arranged in an end to end relationship. One end of the sheath is secured to a control housing and has its interior end in communication with the interior chamber of the housing. The control housing serves to support various control structures for the endoscope including cables which are secured to a terminal tube element with the other ends of the cables secured and looped around a pair of pulleys positioned within the chamber. The pulleys are turned by control knobs to flex a terminal section of the endoscope. The instrument has an optical system with a flexible bundle of optically aligned transparent glass fibers. The transparent glass fibers transmit light from an object which is illuminated by a pair of lamps in the end of the instrument so that an image of the object can be seen at an eyepiece.

U.S. patents issued to Bazinet (U.S. Pat. No. 3,162,214), Takahashi et al. (U.S. Pat. No. 4,236,509) and Petruzzi (U.S. Pat. No. 4,669,172) disclose flexible tubular structures composed of coiled wire and/or tethered circular ring elements which provide for flexibility in tubular structures. Petruzzi discloses a method for fabricating a flexible shaft comprising a spiral cut member having an essentially uniform inside diameter and a tapered linear profile.

U.S. Pat. No. 4,846,153 issued to Berci discloses an intubating video endoscope which includes an elongated sheath member with a selectively controllable bendable section housing an image forming optical system. A generally rigid section includes a control housing. An image transmitting optical system extends throughout the length of the sheath member and terminates adjacent to the image forming system. A light transmitting system also extends throughout the length of the sheath member to the image forming optical system, the rearward end of which is adapted to be operatively connected to a light source.

U.S. Pat. No. 4,949,716 issued to Chenoweth discloses a hand held medical device with a wide range of nasally placed airway tubes to afford better control of airway tubes. A soft flexible tube surrounding a flat spring has a braided wire which is pulled to control the flexing of the airway tube.

U.S. Pat. No. 4,905,666 to Fukuda, U.S. Pat. No. 5,520,222 to Chikama, and JP 5,329,095 to Ogino teach bending devices which use pulleys or chain driven winding mechanisms which are controlled by cranks and knobs. U.S. Pat. Nos. 5,626,553 and 5,667,476 to Frassica et al. use a lever in conjuction with a moveable pulley.

U.S. Pat. No. 5,327,881 to Greene discloses an intubation assisting device having an elongate stylet adapted to fit within a standard endotracheal tube. A flexible bellows region is provided adjacent to the proximal end of the elongate stylet. Flexible optical fibers and illuminating fibers are disposed with the stylet to enable direct viewing by the operator during the intubation process.

U.S. Pat. No. 5,431,152 to Flam et al. teaches an endotracheal intubating instrument with a forward mounted handle. The handle is mounted on a blade for inserting the endotracheal tube into a patient's mouth.

U.S. Pat. No. 6,539,942 to Schwartz et al., hereby incorporated herein by reference in its entirety, describes an endotracheal intubation device having a series of interlinked, truncated ring-like elements disposed along the distal portion of the tube and a handgrip for controlling the degree of bend in the distal end of the device. An imaging device, such as a nasopharyngoscope, can be inserted through the intubation device to visualize the patient's vocal cords during the intubation procedure. The endotracheal intubation device uses a scissors mechanism without pulleys to bend the distal end of the device.

While the related art teach endotracheal intubation devices, there still exists a need for an improved endotracheal device having a curvable portion so as to facilitate the insertion of an endotracheal tube into a patient.

### OBJECTS

Therefore, it is an object of the present invention to provide an improved endotracheal intubation device having a curvable portion.

These and other objects will become increasingly apparent by reference to the following description.

### SUMMARY OF THE INVENTION

The present invention provides a device to facilitate endotracheal intubation of a patient, comprising: a support means; a tubular element, cantilevered at a proximal end from the support means, having an opposed distal end for insertion into the patient's mouth to place an endotracheal tube; a curvable portion of the tubular element disposed adjacent to the distal end of the tubular element; a gripping means attached to the support means and disposed forward of the proximal end of the tubular element, such that a length of the tubular element projects over the gripping means; a control means provided as a component of or adjacent to the gripping means; and a means for moving the curvable portion transmitting a force applied by the user on the control means to curve the curvable portion, wherein when the control portion is manipulated by the user the curvable portion curves into a generally curved configuration in a controlled manner from a less curved configuration and returns to the less curved conformation when the control means is released.

In further embodiments of the device, the curvable portion comprises a series of interconnected ring elements with spaces therebetween. In further embodiments, the curvable portion comprises one or more recesses in the tubular element. In further embodiments, the recesses are provided as slits in the curvable portion. In further embodiments, the recesses are provided as wedge shaped cuts in the curvable portion. In further embodiments, the tubular element is constructed of stainless steel or a shape memory alloy (SMA). In further embodiments, the tubular element is constructed of Nitinol. In further embodiments, the device further comprises an insufflation attachment on the tubular element to clear secretions from the patient's airway and supply oxygenation. In further embodiments, the means for moving the curvable portion is a wire attached to the control means. In further embodiments, the control means is a trigger mounted on the gripping means. In further embodiments, the device further comprises a visualizing means at a proximal end of the device for visualizing of an image of the throat of the patient when the distal end of the tubular element is advanced forward during the endotracheal intubation procedure.

The present invention provides a method of inserting an endotracheal tube into the trachea of a patient comprising: providing a device to facilitate endotracheal intubation of a patient, comprising a support means, a tubular element, cantilevered at a proximal end from the support means, having a distal end for insertion into the patient's mouth to place the endotracheal tube, a curvable portion of the tubular element disposed adjacent to the distal end of the tubular element, a gripping means attached to the support means and disposed forward of the proximal end of the tubular element, such that a length of the tubular element projects over the gripping means, a control means provided as a component of or adjacent to the gripping means, and a means for moving the curvable portion transmitting a force applied by the user on the control means to curve the curvable portion, wherein when the control portion is manipulated by the user the curvable portion curves into a generally curved configuration in a controlled manner from a less curved configuration and returns to the less curved conformation when the control means is released; sliding the endotracheal tube over the tubular element of the device; inserting the distal end of the tubular element with the endotracheal tube into the patient's mouth; manipulating the control means to curve the curvable portion enough so that the distal end of the tubular element can be safely advanced in the throat of the patient; advancing the distal end of the tubular element to place the endotracheal tube into the trachea of the patient; and removing the tubular element from the patient's mouth.

In further embodiments of the method, the curvable portion comprises a series of interconnected ring elements with spaces therebetween. In further embodiments, the curvable portion comprises one or more recesses in the tubular element. In further embodiments, the recesses are provided as slits in the curvable portion. In further embodiments, the recesses are provided as wedge shaped cuts in the curvable portion. In further embodiments, the tubular element is constructed of stainless steel or a shape memory alloy (SMA). In further embodiments, the tubular element is constructed of Nitinol. In further embodiments, wherein the method further comprises the step of clearing secretions from the patient's airway, after inserting the distal end of the tubular element, by means of an insufflation attachment on the tubular element. In further embodiments, the control means is a trigger mounted on the gripping means. In further embodiments of the method, the control means is manipulated by squeezing to curve the curvable portion.

The present invention provides a method of inserting an endotracheal tube into the trachea of a patient comprising: providing a device to facilitate endotracheal intubation of a patient, comprising a support means, a tubular element, cantilevered at a proximal end from the support means, having a distal end for insertion into the patient's mouth to place the endotracheal tube, a curvable portion of the tubular element disposed adjacent to the distal end of the tubular element, a gripping means attached to the support means and disposed forward of the proximal end of the tubular element, such that a length of the tubular element projects over the gripping means, a control means provided as a component of or adjacent to the gripping means, a means for moving the curvable portion transmitting a force applied by the user on the control means to curve the curvable portion, and a visualizing means at a proximal end of the device for visualizing of an image of the throat of the patient, wherein when the control portion is manipulated by the user the curvable portion curves into a generally curved configuration in a controlled manner from a less curved configuration and returns to the less curved conformation when the control means is released; sliding the endotracheal tube over the tubular element of the device; inserting the distal end of the tubular element with the endotracheal tube into the patient's mouth; viewing the image of the throat of the patient on the visualizing means; manipulating the control means to curve the curvable portion enough so that the distal end of the tubular element can be safely advanced in the throat of the patient; advancing the distal end of the tubular element to place the endotracheal tube into the trachea of the patient; and removing the tubular element from the patient's mouth.

In further embodiments of the method, the curvable portion comprises a series of interconnected ring elements with spaces therebetween. In further embodiments, the curvable portion comprises one or more recesses in the tubular element. In further embodiments, the recesses are provided as slits in the curvable portion. In further embodiments, the recesses are provided as wedge shaped cuts in the curvable portion. In still further embodiments, the tubular element is constructed of stainless steel or a shape memory alloy (SMA). In some embodiments, the tubular element is constructed of Nitinol. In further embodiments, the method further comprises the step of clearing secretions from the patient's airway, after inserting the distal end of the tubular element in step (c), by means of an insufflation attachment on the tubular element. In further embodiments, the control means is a trigger mounted on the gripping means. In further embodiments of the method, the control means is manipulated by squeezing in step (e) to curve the curvable portion.

### BRIEF DESCRIPTION OF THE DRAWINGS

FIG. 1 illustrates an environmental perspective view of one embodiment of an endotracheal intubation device 10 in use.

FIG. 1A illustrates a side view of the endotracheal intubation device 10 of FIG. 1.

FIG. 1B illustrates an environmental perspective view of the endotracheal intubation device 10 inserted into an endotracheal tube E prior to use.

FIG. 2 illustrates a side cross-sectional view of the endotracheal intubation device 10.

FIG. 3 illustrates a distal end view of an endotracheal tube stop 75 taken along line 3-3 of FIG. 2.

FIG. 3A is a cross-sectional view of an endotracheal tube stop 75 with an insufflation attachment 301.

FIG. 3B is a cross-sectional view of an endotracheal tube stop 75 with another embodiment of a insufflation attachment 301′ having a liquid port 320.

FIG. 4 illustrates a cross-sectional view of the back of the handle 20 of the endotracheal intubation device 10 taken along line 4-4 of FIG. 1A.

FIG. 5 illustrates a cross-sectional view of the yoke cavity 170 of the endotracheal intubation device 10 taken along line 5-5 of FIG. 2.

FIG. 6 illustrates a cross-sectional view of the lever cavity 150 of the endotracheal intubation device 10 taken along line 6-6 of FIG. 2.

FIG. 7 illustrates a longitudinal cross-sectional view of a first embodiment of the curvable portion 70 and distal end 47, of the distal portion 45 of the endotracheal intubation device 10.

FIG. 7A illustrates a longitudinal cross-sectional view of a second embodiment of the curvable portion 70′ having a series of vertebra 61′.

FIG. 8A illustrates a right side view of the trigger 30. FIG. 8B illustrates a front view of the trigger 30.

FIG. 9A illustrates a top view of the eyepiece swivel 84. FIG. 9B illustrates a left side view of the eyepiece swivel 84.

FIG. 10 illustrates a left side view of the housing 101 of the handle 20 with the cover 135 removed.

FIG. 11 illustrates a cross-section view along line 11-11 of FIG. 10.

FIG. 12 illustrates a cross-sectional view housing 101 of the handle 20 along line 12-12 of FIG. 10.

FIG. 13 illustrates a cross-sectional view housing 101 of the handle 20 along line 13-13 of FIG. 10.

FIG. 14 illustrates a perspective view of an alternate embodiment an endotracheal intubation device 210 according to the present invention having a video system.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “above”, “top” or “up” as used herein refers to a direction or side, respectively of the device corresponding to the top side of the handle.

The term “below”, “bottom” or “down” as used herein refers to a direction or side, respectively, of the device corresponding to the bottom side of the handle and opposed to the top side.

The term “control means” as used herein refers to any mechanism known in the art that a user can manipulate to control bending of the curvable portion. An example of a control means is a trigger as described herein. The term also encompasses such mechanism as buttons, knobs, wheels or the like that can be squeezed, turned, pressed or otherwise manipulated by a user.

The term “curvable portion” as used herein refers to a part of the tubular element which is curvable. In some embodiments, the curvable portion is provided as a portion with cuts (as slits, wedges etc.) as in the two alternate embodiments described in U.S. patent application Ser. No. 11/514,486 to Schwartz et al. hereby incorporated herein by reference in its entirety. In other embodiments, the curvable portion comprises a series of vertebra as described in U.S. Pat. No. 6,539,942 and U.S. patent application Ser. No. 11/230,392 to Schwartz et al., each of which are hereby incorporated herein by reference in their entirety. The curvable portion can also be provided as a bellows or other tubular structures that can be curved which are known in the art.

The term “means for moving the curvable portion” as used herein encompasses any mechanism, including mechanical or electromechanical mechanisms that can transduce movement of the control means to movement of the curvable portion. The term encompasses, but is not limited to mechanisms such as a control wire in conjunction with a rocker arm lever system, as described herein, or a pulley system.

The term “distal” as used herein refers to the end of the device opposed to the proximal end and towards a patient who is to be endotracheally intubated when the endotracheal intubation device is in use. The term “forward” as used herein refers to a direction or position more towards the distal end of the device than a reference point.

The term “gripping means” as used herein refers to any grip such as, but not limited to a pistol type handgrip described herein, that can be gripped by a user to hold the endotracheal intubation device.

The term “insufflation” as used herein refers to blowing a gas, liquid or powder material into an airway of a patient.

The term “left” as used herein refers to a side of the device corresponding to the left side of the handle when viewed from the proximal end.

The term “lever means” as used herein refers to any apparatus known in the art for translating force which comprises one or more rocker arms or other lever systems.

The term “recess” as used herein is a broad term including any indentation, cleft, slit or cut in the tubular element. The term encompasses “cuts” including narrow cuts as “slits” and also wide V-shaped cuts as “wedges” in the tubular element. “Slits” are provided as narrow cuts preferably disposed on opposed sides of the curvable portion.

The term “proximal” or “back” as used herein refers to a direction towards a medical professional when the endotracheal intubation device is in use.

The term “right” as used herein refers to a side of the device corresponding to the right side of the handle when viewed from the proximal end.

The term “ring elements” as used herein refers to any set of ring shaped structures, that when arranged in a series can be curved. Ring elements can be of a structure including, but not limited to, the vertebra as described in U.S. Pat. No. 6,539,942 and U.S. patent application Ser. No. 11/230,392 to Schwartz et al.

The term “SMA” as used herein is an abbreviation for a shape memory alloy, also known as a memory metal or smart wire. Some examples of SMAs include, but are not limited to copper-zinc-aluminum, copper-aluminum-nickel, and nickel-titanium (NiTi) alloys such as Nitinol (Nickel Titanium Navy Ordnance Labs).

The term “support means” as used herein refers to any structure capable of supporting the tubular element of the device, so that a length of the tubular element projects over the gripping means.

The term “tubular element” as used herein refers to an elongate member, including but not limited to a tubular element described herein. The tubular element can be cylindrical, however it is not limited thereto. Any elongate shape that an endotracheal tube can slide over is encompassed by the present invention.

The term “transmission means” as used herein refers to a any mechanism or device for transmitting an image of the throat from the viewing means to the visualizing means. An example of a transmission means includes, but is not limited to, electrical wiring lines, fiber optic lines, and/or optical lenses.

The term “viewing means” as used herein refers to any mechanism or device for collecting an image of the throat of the patient at the distal end of the tubular element during the endotracheal intubation procedure. An example of a viewing means includes, but is not limited to, a small video camera or a lens for a fiber optics system.

The term “visualizing means” as used herein refers to any mechanism or device for visualizing or displaying an image of the throat of the patient at the distal end of the tubular element during the endotracheal intubation procedure. An example of a visualizing means includes, but is not limited to, a liquid crystal display or other type of video display. Other examples included one or more lenses which collect an image from a fiber optics system to be viewed in an eyepiece.

U.S. patent application Ser. Nos. 11/230,392 and 11/514,486 to Schwartz et al., hereby incorporated herein by reference in their entirety, describe endotracheal intubation devices. The present invention is an improvement of these devices. The present invention provides a device to facilitate endotracheal intubation of a patient. The device comprises: a support means; a tubular element, that is cantilevered at a proximal end from the support means, having an opposed distal end for insertion into the patient's mouth to place an endotracheal tube. The device further comprises a curvable portion of the tubular element disposed adjacent to the distal end of the tubular element. A gripping means that is attached to the support means is disposed forward of the proximal end of the tubular element, such that a length of the tubular element projects over the gripping means. A control means is provided as a component of or adjacent to the gripping means as well as a means for moving the curvable portion transmitting a force applied by the user on the control means to curve the curvable, portion. When the control portion is manipulated by the user the curvable portion curves into a generally curved configuration in a controlled manner from a fully straight or less curved configuration and returns to the less curved conformation when the control means is released.

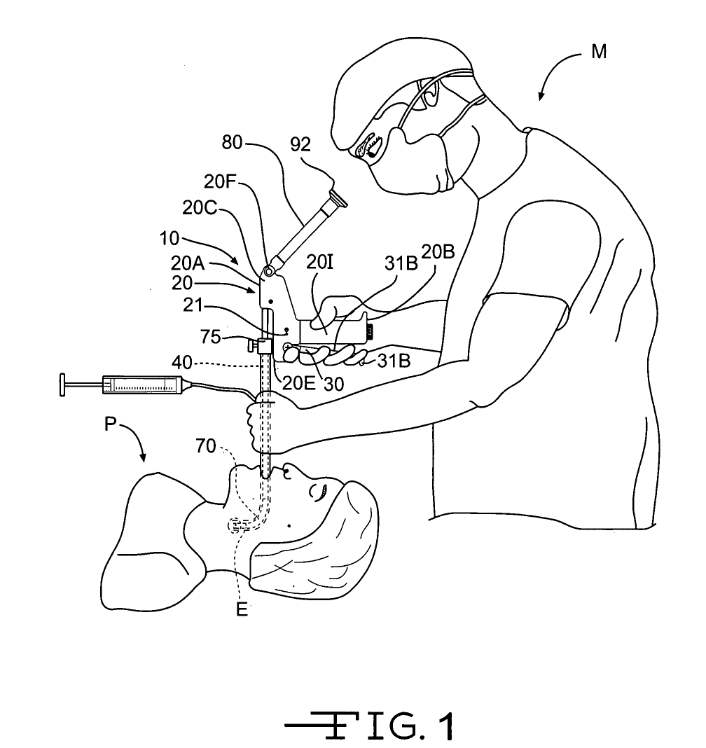

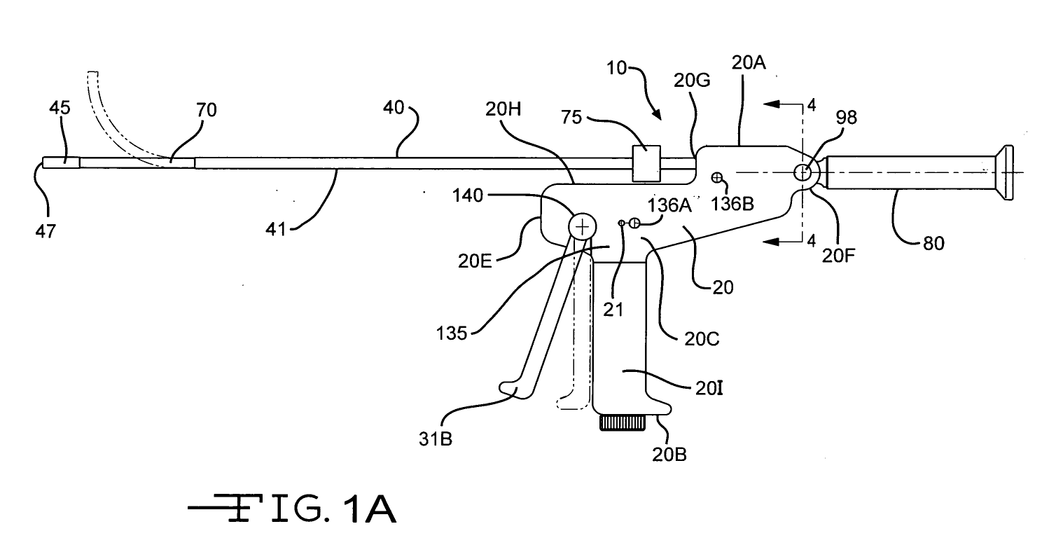

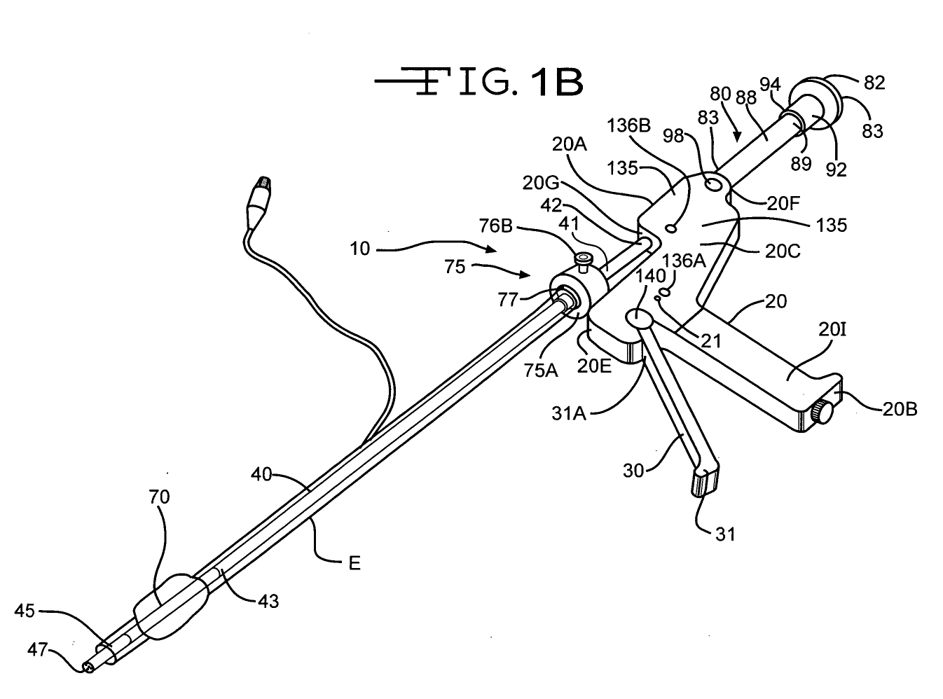

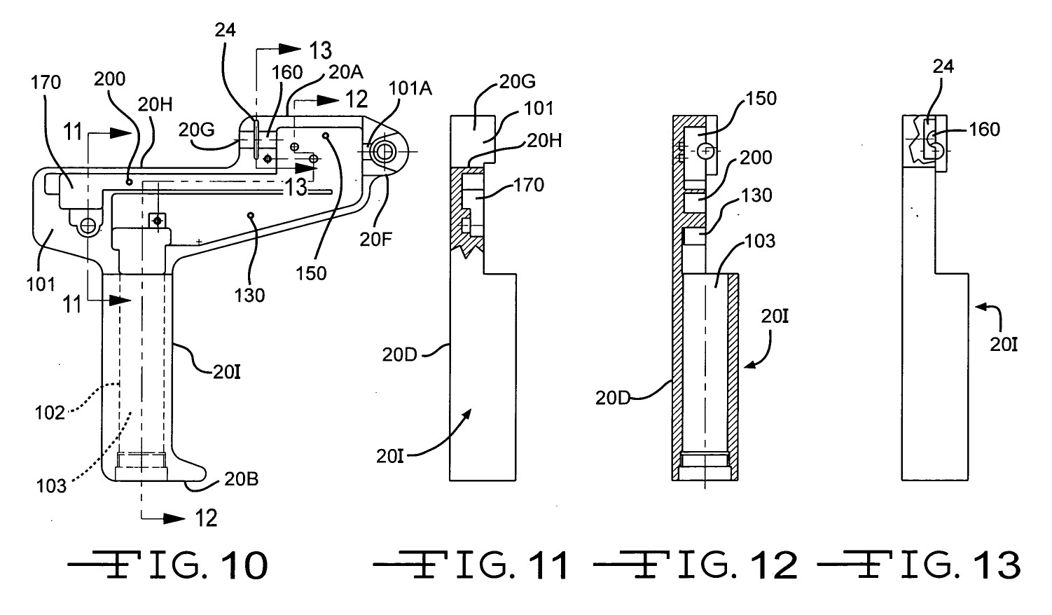

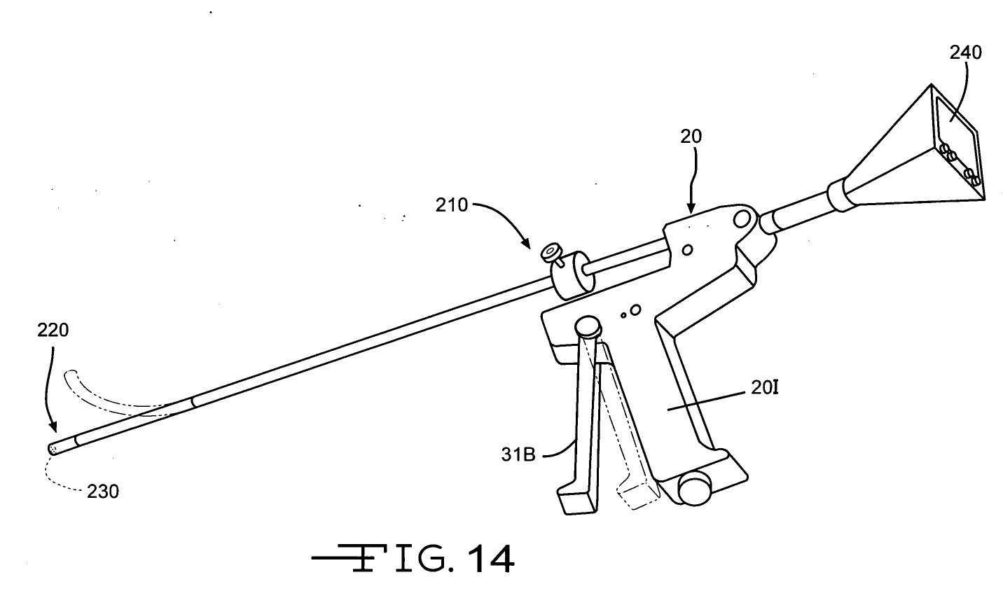

One embodiment of the present invention is illustrated in FIGS. 1 through 13. A side view of this embodiment of the endotracheal intubation device 10 is illustrated in use in FIG. 1. FIG. 1 shows how a tubular element 40 of the endotracheal intubation device 10 can be inserted into a patient P by a medical professional M. As illustrated in FIG. 1A, the handle 20 has an upper top surface 20A, lower top surface 20H, a bottom 20B, a left 20C, a right 20D (FIGS. 11, 12, and 13), a front 20E, a back 20F, and a mounting surface 20G. A portion of the top of the handle 20 projects up to form the upper top surface 20A, adjacent to the back 20F of the handle 20. On the front of this projecting portion of the handle 20 is a mounting surface 20G. Forward of the mounting surface 20G is a lower portion of the handle 20 as defined by the lower top surface 20H. The tubular element 40 attaches at a proximal end 42 of a proximal portion 41 of the tubular element 40 at the mounting face 20G on the portion of the top of the handle 20 that extends upward near the back 20F. The tubular element 40 is cantilevered from the mounting surface 20G and extends over a step formed by the lower top surface 20H. The tubular element 40 extends forward over the front 20E of the handle 20 to a distal end 47 of a distal portion 45 of the tubular element 40. The distal end 47 of the tubular element 40 is inserted into the patient's throat to place the endotracheal tube E into the patient. The proximal end of the endotracheal tube E can extend back more proximal than the handgrip 20I. This configuration makes it easier to manipulate than a longer endotracheal intubation device, since the hand of the medical professional or other user is closer to the distal end 47 of the device and thus the end of the endotracheal tube E which is inserted into the patient. Since the hand of the user grips closer to the distal end 47 of the device, any forces applied on the distal end 47 causes less twisting in the hand of the user. The configuration also makes it easier for shorter users.

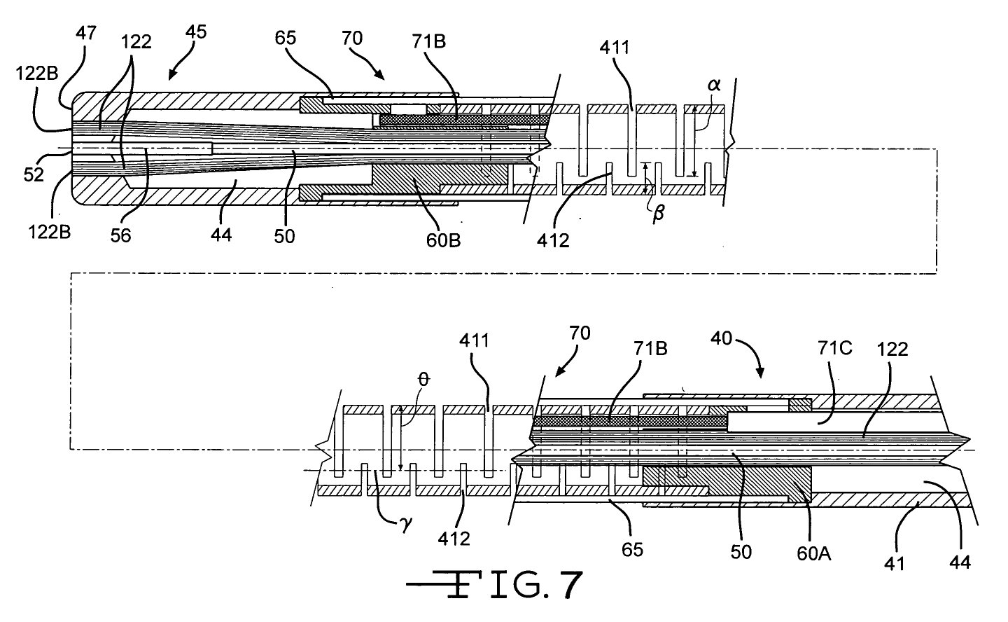

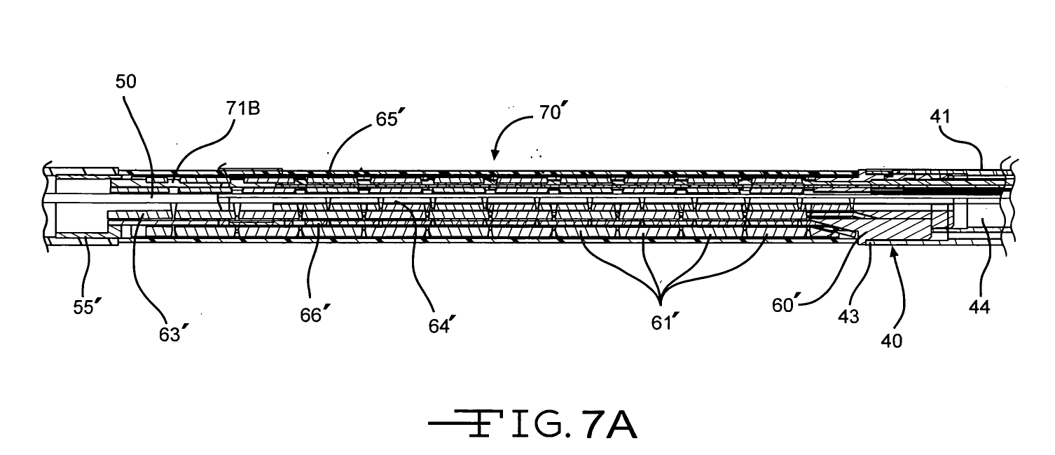

The handle 20 of the endotracheal intubation device 10 is gripped by the medical professional M (FIG. 1) on a handgrip 20I to insert the device 10. The fingers of the medical professional M are used to grip a trigger 30 which is pivotably mounted at a top end 31A to the handle 20. The trigger 30 has a bottom portion 31B for controlled movement when the medical professional M squeezes the trigger 30 towards the handgrip 20I of the handle 20. When the medical professional M squeezes the bottom portion 31B of the trigger 30 a curvable portion 70 (FIGS. 1, 1A, 1B and 7) towards the distal end of the tubular element 40 is curved upward into a generally curved configuration in a controlled manner from a fully straight or less curved configuration. The medical professional M can thereby move the distal end 47 of the tubular element 40 to safely advance the tubular element 40 into the throat of the patient P to insert an endotracheal tube E. The endotracheal intubation device 10 is well sealed so that bodily fluids cannot penetrate the device 10 and damage any internal components.

FIG. 1B illustrates how the endotracheal tube E is inserted over the tubular element 40 of the endotracheal intubation device 10 to a stop 75 (FIGS. 1, 1A, 1B, 2 and 3) prior to using the device 10 to endotracheally intubate the patient. The adjustable endotracheal tube stop 75 (FIGS. 1B, 2 and 3) is placed over the tubular element 40 to encircle the proximal portion 41. The endotracheal tube stop 75 has a first end 75A having a first circular opening 75C with a diameter adapted to receive an end of a standard adapter 77 on an endotracheal tube E as illustrated in FIG. 1B. An o-ring 75E (FIG. 2) in the endotracheal tube stop 75 grips the standard adapter 77 (FIG. 1B) at the end of the endotracheal tube in the first opening 75C. A first stop screw 76A can be tightened so that the endotracheal tube E will not slide off during the intubation procedure. At a second end 75B of the endotracheal tube stop 75 is a second opening 75D having a diameter fits over the tubular element 40. The endotracheal tube stop 75 is secured in place on the proximal portion 41 by means of a second stop screw 76B, so that the endotracheal tube does not slide with respect to the tubular element 40 during the intubation procedure. The stop 75 can be slid to a more proximal location towards the back of the device, so that the handgrip 20I is forward of the proximal end of the endotracheal tube E.

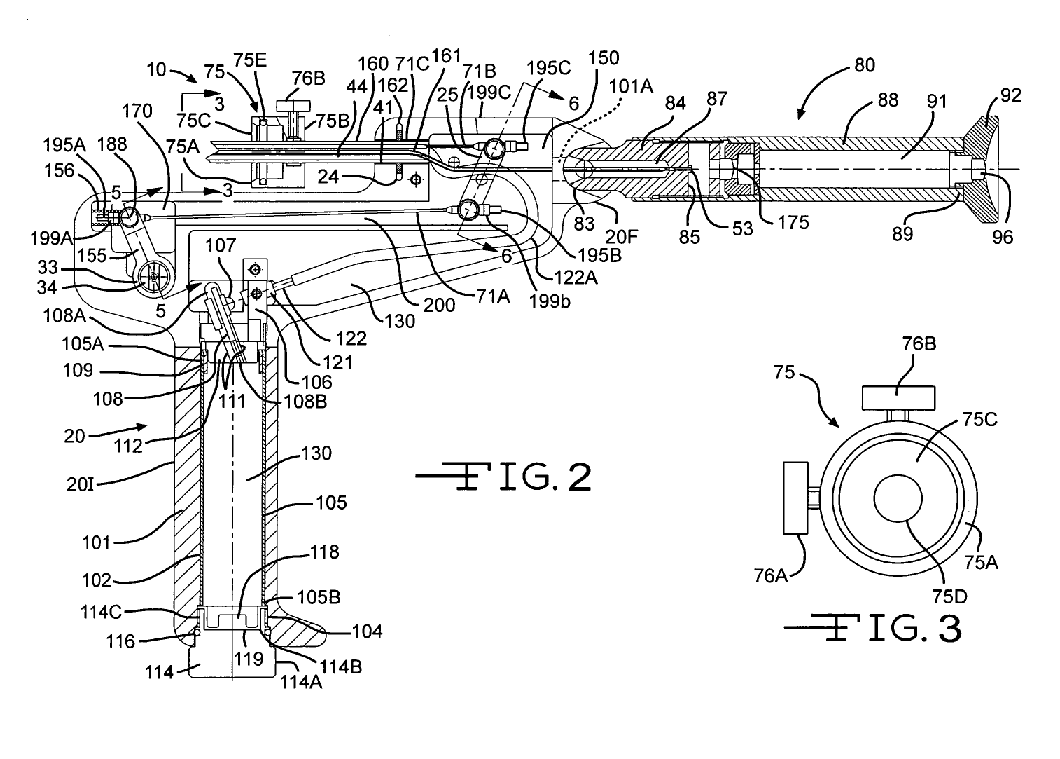

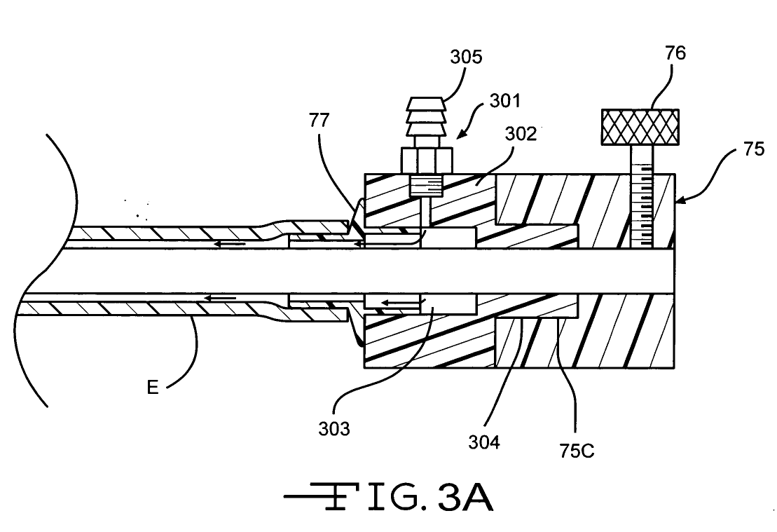

In some embodiments, an insufflation attachment 301 described in U.S. patent application Ser. No. 11/514,486 to Schwartz et al. can be inserted between the standard adapter 77 of an endotracheal tube E and the endotracheal tube stop 75. As illustrated in FIGS. 3A and 3B, an insufflation attachment (301, 301′) can be inserted between the standard adapter 77 and the endotracheal tube stop 75. A wide portion (302, 302′) of the insufflation attachment (301, 301′) has a cavity (303, 303′) into which the standard adapter 77 fits. At an opposing end of the insufflation attachment (301, 301′) a narrow portion (304, 304′) projects from the wide portion (302, 302′) having an outer diameter that fits into the first opening 75C at the first end 75A of the endotracheal tube stop 75 and an inner diameter that fits over the tubular element 40. In some embodiments, the insufflation attachment (301, 301′) is disposable. The insufflation attachment (301, 301′) can be hooked up to an oxygen source tubing placed by means of a port (305, 305′) passing through the wide portion (302, 302′) and into the cavity (303, 303′) to allow oxygen to flow into and through the endotracheal tube E to clear secretions from the patient's airway and supply oxygenation. Optionally, in one embodiment of the insufflation attachment 301′, as illustrated in FIG. 3B, a liquid port 320 can be added as well to allow injection of a local anesthetic drug. A drug, for example an aminoester or aminoamide local anesthetic such as lidocaine can be administered through the liquid port 320 so that it passes down the endotracheal tube E and into the patient. In some embodiments the liquid port 320 is provided as a luer-lock type attachment so that a syringe (not shown) can be easily attached to the insufflation attachment 301′. A removable cap 321 is provided to seal the liquid port 320 when not in use.

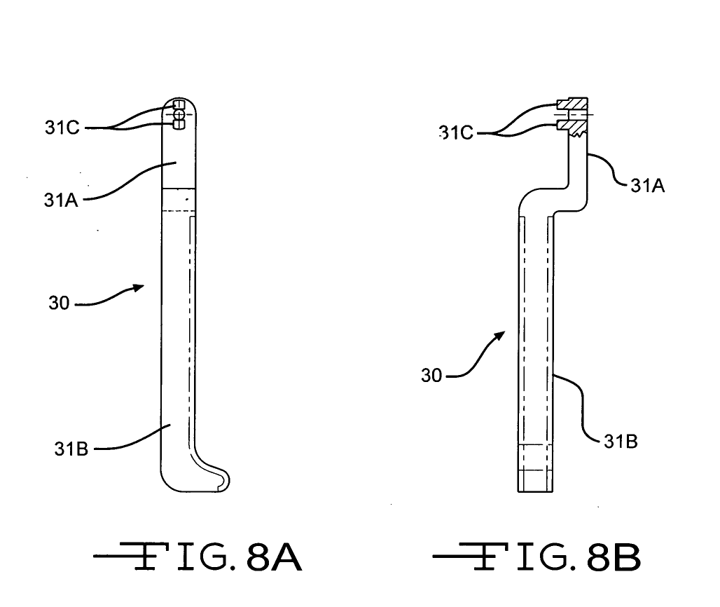

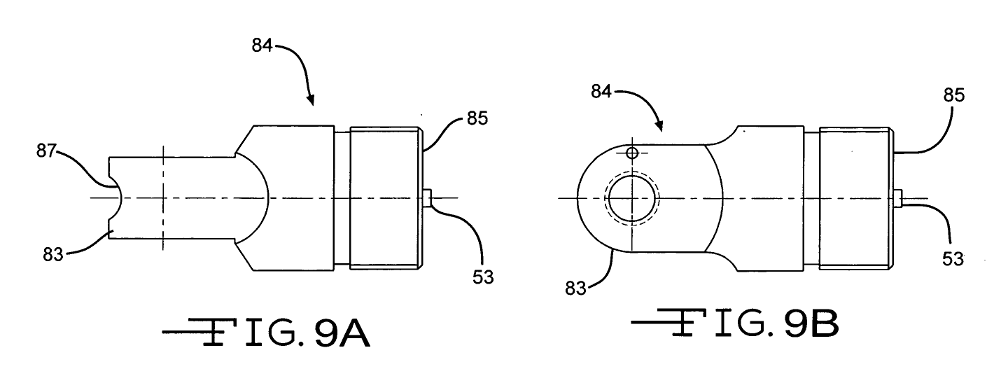

As seen in FIG. 2, a pivotable optics portion 80 is attached to the back 20F of the handle 20 on an eyepiece swivel 84 (illustrated in FIGS. 9A and B) at the distal end 83 of the optics portion 80. An eyepiece tube 88 projects from a proximal end 85 of the eyepiece swivel 84. At the proximal end of the optics portion 80 is an eyepiece housing 92 which is mounted over the proximal end 89 of the eyepiece tube 88. As illustrated in FIG. 1, the medical practitioner M can look into the eyepiece housing 92 of the optics portion 80 to view an image of the patient's throat as the distal end 47 (FIG. 7) of the tubular element 40 is advanced to place the endotracheal tube E in the trachea of the patient P. Since the endotracheal intubation device 10 incorporates internal optics, it can be used in situations where an external imaging device, such as a nasopharyngoscope, is not readily available.

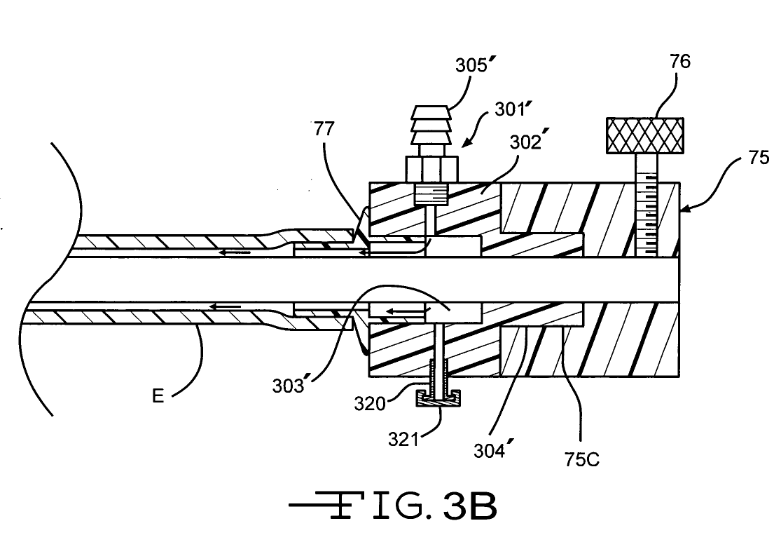

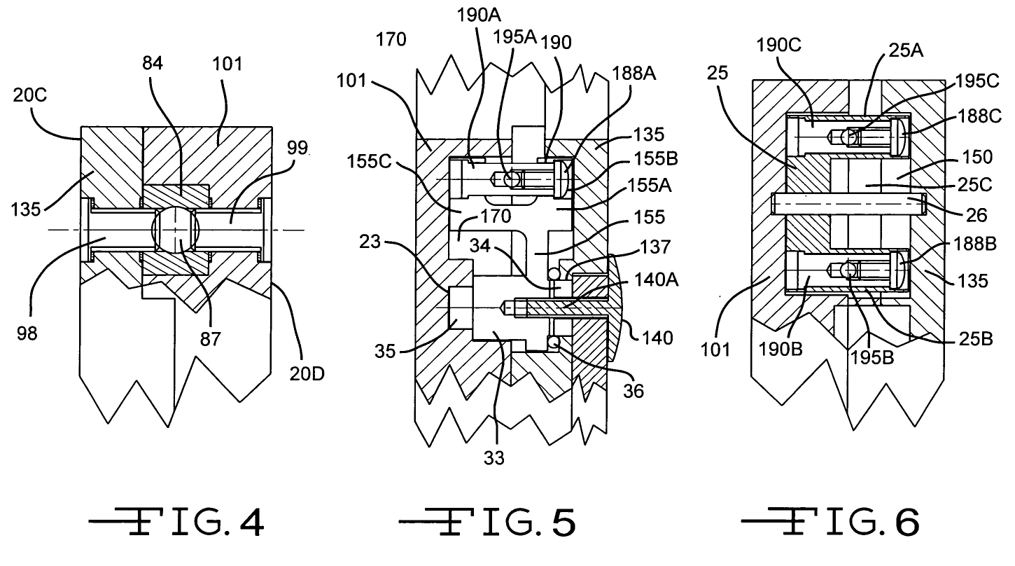

FIG. 2 also illustrates the internal workings of the endotracheal intubation device 10 in detail. Within the handle 20, as best seen in FIG. 10, an illumination fiber cavity 130, lever cavity 150, yoke cavity 170 (FIG. 11) and a control wire cavity 200 are enclosed by a housing 101 on the right side and the cover 135 on the left side as illustrated in FIGS. 5 and 6. As illustrated in FIGS. 1A and 1B, the cover 135 is attached to the housing 101 by a trigger cap 140 (FIG. 5), a front screw 136A, a back screw 136B and a left pivot screw 98 (FIG. 4) at a back of the cover 135. In the handgrip 20I of the handle 20, the housing 101 has an inner wall 102 which defines a battery cavity 103. As seen in FIG. 2, the battery cavity 103 encloses a cylindrical battery sleeve 105 constructed of brass or other conducting material, having a top end 105A and a bottom end 105B. The sleeve 105 is disposed against the inner wall 102 of the housing 101 of the handgrip 20I. Two batteries (not shown) can be inserted in a series conformation within the cylindrical sleeve 105 in the battery cavity 103. The two batteries are held in the battery cavity 103 by a battery plug 114 mounted below the two batteries at the bottom 20B of the handle 20. The battery plug 114 has a first portion 114A which can be gripped when inserting the battery plug 114 into the battery cavity 103 after insertion of the batteries. A battery plug o-ring 116 surrounds a second portion 114B in the center of the battery plug 114 and rests snugly against the inner wall 102 of the housing 101 of the handle 20 when the battery plug 114 is inserted. The battery plug 114 has a third portion 114C with a thread which is screwed into a threaded portion 104 in the inner wall 102 of the battery cavity 103. Since the battery plug o-ring 116 fits snugly against the inner wall 102, the battery plug 114 will not loosen when the device 10 is in use. The battery plug o-ring 116 also keeps fluid out of the device 10. A contact cap 119 fits into a depression in the third portion 114C of the battery plug 114. When the battery plug 114 is threaded into the handle 20, a spring (not shown) which is disposed over a projection 118 in the contact cap 119 is held against a negative terminal of a lower of the batteries to make electrical contact and support the batteries in the battery cavity 103.

Enclosed above the sleeve 105 in the battery cavity 103 is a lamp housing 106. A lamp 107 which is the light source for the endotracheal intubation device 10 is mounted in a lamp mount 108 in the lamp housing 106. In this embodiment, the lamp 107 is provided as a light-emitting diode (LED), while in alternative embodiments the lamp 107 can be a xenon lamp or other similar high-intensity light source. The LED lamp 107 is affixed to a back side of the top 108A of the lamp mount 108, so as to project light back to a first end of a plurality of illumination fibers 122 held in the lamp housing 106. The top 108A of the lamp mount 108 angles forward and attaches at a bottom 108B to an insulator cap 112 that is inserted in an insulator ring 112A supporting a contact 109 at the top end 105A of the battery sleeve 105. The insulator ring 112A and insulator cap 112 secure the lamp mount 108 to the sleeve 105 while also isolating the contact 109 and sleeve 105 from electrical connection with the positive terminal of the batteries at the top of the battery cavity 103. The leads 111 from the lamp 107 make electrical contact with the contact 109 on the sleeve 105 and the positive terminal of one of the batteries (not shown) at the top of the battery cavity 103. In the housing 101, adjacent to the lamp 107, is a transparent window 21 in the cover 135 as seen in FIGS. 1, 1A, and 1B. The transparent window 21 is mounted in an opening that penetrates the left side 20C of the handle 20 and the lamp apparatus 106 over the lamp 107. When the lamp 107 is turned on the transparent window 21 is lit as a reminder that the power is on. It is to be understood that other battery configurations and other power sources known in the art can be used in other embodiments of the device, instead of the battery system described herein, to power the lamp 107.

As illustrated in FIG. 2, the lamp housing 106 is penetrated from behind the LED lamp 107 by a fiber ferrule 121 into which the proximal ends of illumination fibers 122 are secured. The illumination fibers 122 are held by the fiber ferrule 121 in close proximity to the LED lamp 107, so that the illumination fibers 122 can collect the light from the lamp 107 when it is powered by the batteries. In use, the lamp 107 can be activated by turning the battery plug 114, so as to advance the battery plug 114 into the battery cavity 103 until the contact cap 119 makes electrical contact with the bottom end 105B of the sleeve 105. This action completes an electrical circuit so as to supply power to the lamp 107. The lamp 107, when supplied with power, emits light adjacent to the fiber ferrule 121, where the light is collected by the proximal end of the illumination fibers 122. The illumination fibers 122 extend from the fiber ferrule 121 in the lamp housing 106 into an illumination fiber cavity 130. The illumination fibers 122, covered by a protective tubing 122A, extend back into the illumination fiber cavity 130 and up into a lever cavity 150 above the back end of the illumination fiber cavity 130 at the back 20F of the handle 20. The illumination fibers 122, covered by the protective tubing 122A then pass from the lever cavity 150 into an internal channel 44 of the tubular element 40 at the proximal end 42.

The internal channel 44 (FIGS. 2 and 7) in the tubular element 40 extends the length of the tubular element 40 from the proximal end 42 which opens into the lever cavity 150, through the proximal portion 41 of the tubular element 40, the curvable portion 70, and through the distal portion 45 to the distal end 47, as illustrated in FIG. 7. The illumination fibers 122, covered by the protective tubing 122A pass from the lever cavity 150 into and through the internal channel 44 of the tubular element 40 to the distal end 47 of the device where they terminate at the distal ends 122B as illustrated in FIG. 7. The illumination fibers 122 thereby carry light from the LED lamp 107 collected at the proximal ends in the fiber ferrule 121 to the distal end 47 of the device 10. When the battery plug 114 is turned to complete the circuit and provide power to the lamp 107, the throat of the patient is illuminated with light from the distal end 122B of the illumination fibers 122.

As best seen in FIG. 7, a tube 56 extends from the distal head 48 inside of the distal portion 45 to support a first end 52 of an optics fiber 50. A lens (not shown) for the optics fiber 50 is located between the distal end 122B of the illumination fibers 122. In one embodiment, the lens in the aperture has approximately a 60° field of view. The optics fiber 50 extends from the lens (not shown) at a first end 52 of the optics fiber 50 and passes through the length of the internal channel 44 to a second end 53 in the eyepiece swivel 84 near the back 20F of handle 20. The optics fiber 50 extends from the internal channel 44 of the tubular element 40 through the lever cavity 150 and through an optics opening 101A which penetrates the housing 101 at the back 20F of the handle 20. The optics fiber 50 then passes into an optics portion 80 of the device 10.

As seen in FIG. 4, a eyepiece swivel 84 (FIGS. 9A and B) is mounted at a left side on the left pivot screw 98 which penetrates the cover 135 at the back 20F and left 20C of the handle 20. In like fashion, the eyepiece swivel 84 is mounted at a right side on a right pivot screw 99 penetrating the housing 101 at the back 20F and right 20D of the handle 20. The left pivot screw 98 and the right pivot screw 99 do not obstruct the internal channel 87 of the eyepiece swivel 84. Therefore, the optics portion 80 can be moved up and down with respect to the handle 20 although the optics fiber 50 extends through to the proximal end 85 of the eyepiece swivel 84. The optics fiber 50 passes through the optics opening 101A in the back of the handle 20 and passes through the eyepiece swivel 84 at the distal end 83 of the optics portion 80. The second end 53 of the optics fiber 50 passes through the internal channel 87 of the eyepiece swivel 84 and terminates at a proximal end 85 of the eyepiece swivel 84.

As illustrated in FIG. 2, an eyepiece tube 88 attaches to the proximal end 85 of the eyepiece swivel 84 having an internal channel 91 which extends from the eyepiece swivel 84 (FIG. 4) to the proximal end 89 of the eyepiece tube 88. An eyepiece housing 92 is threaded over the eyepiece tube 88, which in some embodiments is an 18 mm Ortho eyepiece. The eyepiece housing 92 flares outward to provide a circular lip used as an eye rest. At the proximal end of the eyepiece housing 92 is an opening 96 centrally located in the eyepiece housing 92. The optics portion 80 focuses light collected at the lens of the optics fiber 50 by means of one or more lenses 175 from light which is emitted from the second end 53 of the optics fiber 50. The lenses 175 of the optics portion 80 are configured so that an image of the throat of the patient can be viewed through the opening 96 in the eyepiece housing 92. An image of the throat of the patient can be viewed through the opening 96 in the eyepiece housing 92 when the distal end 47 of the tubular element 40 is advanced forward during the endotracheal intubation procedure.

An alternative embodiment of the present invention is shown in FIG. 14 which is identical to the embodiment of FIGS. 1 through 13, except for the optics system. In this embodiment, a small video camera 220 at the distal end 230 of the device 210 is wired through to a video display 240 mounted on the proximal end of the device 210. In this embodiment, the handle 20 is as described previously, however when the medical professional grips the handgrip 20I and squeezes the bottom end 31B of the trigger, the throat of the patient is viewed on the video display 240. In further embodiments, a small video display mounted in a proximal end of the device can be viewed through an opening in the eyepiece housing when the distal end of the tubular element is advanced forward during the endotracheal intubation procedure. The video display can also be provided separate from the endotracheal device of the present invention.

As illustrated in FIG. 5, a yoke cavity 170 at the front of the handle 20 is enclosed by the housing 101 on the right side and the cover 135 on the left side. As described previously, the trigger 30 which is mounted on the handle 20 has a bottom portion 31B for controlling the degree of bend of the curvable portion 70 of the tubular element 40 when the trigger 30 is squeezed towards the handle 20. The top portion 31A of the trigger 30 is mounted to a pivot portion 33 (FIGS. 2 and 5) of a yoke 155 mounted within the yoke cavity 170 of the handle 20. As illustrated in FIG. 5, the pivot portion 33 extends laterally right to left across the handle 20. The pivot portion 33 extends from a right mounting post 35 rotatably mounted in a right mounting hole 23 in the inner wall at the right side 20D of the handle 20, to a left mounting post 34 which is rotatably mounted in a left mounting hole 137 in the cover 135 at the left side of the handle 20. As seen in FIG. 5, a pivot o-ring 36 fits around the left mounting post 34 between the pivot portion 33 and the cover 135 surrounding the left mounting hole 137. The top end 31A of the trigger 30 has two posts 31C (FIGS. 8A and B) that pass through the left mounting hole 137 in the cover 135 and fit in a slot 34A in the left mounting post 34. A trigger pivot pin 140A on the trigger cap 140 penetrates a first pivot pin hole 141 through the top end 31 of the trigger 30 and a second pivot pin hole 34A in the slot 34A in the left mounting post 34 of the pivot portion 33 to secure the top end 31 of the trigger 30 to the pivot portion 33 of the yoke 155.

As illustrated in FIG. 5, a left projection 155A and a right projection 155C of the yoke 155 extend upwards to define a space through which a cylindrical yoke swivel 190A is mounted. A hollow wire fitting 195A is mounted inside the cylindrical yoke swivel 190A. A length adjacent to a second end 73 of a first control wire 71A (FIG. 2) is secured inside the wire fitting 195A so that the first control wire 71A extends from the wire fitting 195A and through a control wire cavity 200 towards the back 20F of the handle 20 and into the lever cavity 150. As seen in FIG. 2, two jam nuts 199A are threaded and locked over an external thread on the wire fitting 195A and rest against the yoke swivel 190A (FIG. 5) towards the front 20E of the handle 20. As seen in FIG. 5, a yoke pin 188A penetrates a hole 155B through the left projection 155A of the yoke 155 and threaded into a left hole in the yoke swivel 190A. In a similar manner, the yoke swivel 190A penetrates a hole 155D through the right projection 155C of the yoke portion 155. This allows the assembled pieces to swivel in the yoke portion 155 when the yoke portion 155 moves forward and backward in the yoke cavity 170. When the bottom end 31B of the trigger 30 is squeezed towards the handle 20 the pivot portion 33 and the yoke portion 155 rotates forward so as to act as a lever. A tension spring 156 is mounted over the two jam nuts 199A and wire fitting 195A, and rests between the yoke portion and a front wall 170A of the yoke cavity 170. The tension spring 156 resists forward movement of the yoke portion 155, and helps the yoke portion 155 to return backward again when pressure on the bottom end 31B of the trigger 30 is released.

As seen in FIG. 2, the first control wire 71A extends from the yoke cavity 170 at the front of the handle 20, through the control wire cavity 200, and into the lever cavity 150 at the back 20F of the handle 20. As seen in FIGS. 2 and 6, the lever cavity 150 of the housing 101 encloses a rocker arm 25 which is mounted on a rocker arm pin 26 which extends from a right side mounted in the housing 101 and a left side which is mounted in the cover 135 to form one embodiment of a lever means. As illustrated in FIG. 6, a top arm 25A and a bottom arm 25B of the rocker arm 25 extend horizontally from the rocker arm 25 so as to define a space 25C through which the optics fiber 50 and illumination fibers 122 pass. In a similar manner to the yoke 155 described above, the first control wire 71A and the second control wire 71B attach to the bottom arm 25A and the top arm 25C, respectively, by means of cylindrical rocker arm swivels 190B and 190C. The first control wire 71A and second control wire 71B are attached by means of hollow wire fittings 195B, 195C that are mounted inside the rocker arm swivels 190B and 190C. A length adjacent to an end of the first and second control wires 71A, 71B are secured inside the bottom wire fitting 195B and top wire fitting 195C, respectively. As seen in FIG. 2, two sets of jam nuts 199B, 199C are threaded and locked over external threading on the wire fittings 195B, 195C and rest against the swivels 190B, 190C towards the back of the handle 20. Yoke pins 188B, 188C are threaded into holes in the rocker arm swivels 190B, 190C. This allows the assembled pieces to swivel in the rocker arm 25 when it rotates forward and backward in the lever cavity 150. The second control wire 71B extends into a wire support tube 71C (FIGS. 2, 7) in the internal channel 44 of the tubular element 40 at the proximal end 42 of the proximal portion 41. The second control wire 71B passes through the wire support tube 71C in the internal channel 44 of the tubular element 40 and exits the wire support tube 71C adjacent to the curvable portion 70 of the tubular element, as seen in FIG. 7. The control wire 71 extends along the top of the curvable portion 70 and attached to a distal mount 60B at the distal end 70B of the curvable portion 70.

As illustrated in FIGS. 2, 10, and 13, a mounting channel 160 extends a length through the housing 101 and the cover 135 of the handle 20. The mounting channel 160 extends from the mounting face 20G of the handle 20 to the lever cavity 150. Mounted flush with the wall defining the mounting channel 160 and extending the length of the mounting channel 160 is a tubular mounting shaft 161. The mounting shaft 161 is anchored in the handle 20 by means of a central rim 162 which encircles the mounting shaft 161 (FIG. 2) and fits into a slot 24 (FIGS. 2, 10, 13) in the handle 20 and the cover 135 to enclose the central rim 162 (FIG. 2). The proximal end 42 of the proximal portion 41 of the tubular element 40 has an internal radius such that it fits tightly within the tubular mounting shaft 161. The tubular element 40 extends through the length of the mounting shaft 161 to secure the tubular element 40 to the handle 20 at the mounting face 20G. Since the rocker arm 25 mechanism makes it possible to locate the lever cavity 150 at the back 20F of the handle 20, the mounting face 20G can be located behind the handgrip 20I. This configuration of the device allows the proximal portion 41 of the tubular element 40 to be cantilevered over the lower top surface 20H and handgrip 20I. Since, this places the handgrip 20I forward of a proximal end of the endotracheal tube E, manipulation of the endotracheal tube E is more manageable, with less twisting forces on the user's hand.

When it is clear from the image of the throat of the patient that the distal end 47 of the tubular element 40 must be curved to avoid throat structures such as the back of the throat, the trigger 30 can be squeezed to curve the curvable portion 70 to then view the vocal cords. A first embodiment of the curvable portion 70 is illustrated in FIG. 7. As described previously, the illumination fibers 122, the optics fiber 50 and the second control wire 71B pass through the curvable portions 70. Slits 411, 412 are made, optionally by means of a laser cutting device, in the tubing to allow the curvable portion 70 to bend when the trigger is squeezed. In the embodiment illustrated in FIG. 7, two sets of alternating slits (411, 412) are provided in the curvable portion 70 of the tubular element 40. A first set of slits 411 are on a top side of the curvable portion 70, and a second set of slits 412 are on the bottom side of the curvable portion 70. The first set of cuts are deeper than the second set to allow bending of the curvable portion in an upward direction. In one embodiment, the first set of cuts 411 are of a depth α of 0.18 inches (4.57 mm) with a width of 0.015 inches (0.3.8 mm). In this embodiment, the second set of cuts 412 are of a depth β of 0.0675 inches (1.71 mm) with a width of 0.015 inches (0.38 mm). In this embodiment, the cut separation is 0.035 inches (0.89 mm). The length of the curvable portion 70 in this one embodiment is 2.6 inches (66 mm). As seen in FIG. 7, the depth α of the first set of cuts 411 and the depth β of the second set of cuts 412 overlap such that the curvable portion 70 bends along a bend line γ at a distance θ from the top of the curvable portion 70. In some preferred embodiments, the distance θ is about ⅔ of the width of the tubular element 40.

The proximal end of the curvable portion 70 is mounted in the proximal portion 41 of the tubular element 40 by means of a proximal mount 60A. The entire length of the curvable portion 70 is covered with a protective tubing 65, such as Viton® tubing (DuPont, Wilmington, Del.) or other robust tubing material which seals the internal components of the curvable portion 70 through which the optics fiber 50 and the illumination fibers 122 extend. As described previously, the second control wire 71B extends to a distal mount 60B at the distal end of the curvable portion 70 where the second control wire 71B is secured. When the bottom end 31B of the trigger 30 is squeezed, the top end 32 of the trigger 30 rotates the yoke 155, which acts as a lever to pull the second end 73 of the first control wire 71A. The first control wire 71A rotates the rocker arm 25 so that tension is applied to the second control wire 71B. The tension on the second control wire 71B curves the curvable portion 70 in a controlled manner from a fully straight or less curved configuration to a more curved configuration.

While the tubular element 40 can be constructed of stainless steel, polymer or other sturdy material, in some preferred embodiments the curvable portion 70 is constructed of a shape memory alloy (SMA). Any shape memory alloy such as a copper-zinc-aluminum, copper-aluminum-nickel, and nickel-titanium (NiTi) alloys can be used, such as, but not limited to Nitinol. The shape memory alloy (SMA) of the curvable portion 70 will flex when the trigger 30 is squeezed, and then will return to its original conformation when the trigger 30 is released due to the tendency of the SMA to spring back to a less curved conformation. The curvable portion can be provided as a portion with cuts (as slits, wedges etc.) as described above. Some alternate embodiments are described in U.S. patent application Ser. No. 11/514,486 to Schwartz et al. hereby incorporated herein by reference in its entirety. The curvable portion can be provided as a separate component or as a continuous piece with the rest of the tubular element. It is to be understood that the curvable portion can also be provided as a bellows or other tubular structures that can be curved which are known in the art.

In other embodiments, the curvable portion can comprise a series of vertebra 61′ as illustrated in FIG. 7A and described in U.S. Pat. No. 6,539,942 and U.S. patent application Ser. No. 11/230,392 to Schwartz et al., each of which are hereby incorporated herein by reference in their entirety. As illustrated in FIG. 7A, the curvable portion 70′ is mounted on a vertebra mount 60′ to a distal end 43 of the proximal portion 41 of the tubular element 40. The entire length of the curvable portion 70′ is covered with a protective tubing 65′ such as Viton® tubing (DuPont, Wilmington, Del.) or other robust tubing material which seals the series of vertebra 61′ and other internal components of the curvable portion 70′. The series of vertebra 61′ are mounted at a proximal end of the curvable portion 70′ by means of the vertebra mount 60′ and at a distal end by a distal mount 55′. A fiber cavity 64′ provides an extension of the internal channel 44 of the tubular element 40 through which the optics fiber 50 and the illumination fibers (not shown) extend. A wire rope 66′ extends from the proximal mount 60′ and sequentially passes through each vertebra 61′ in the series and finally to the first vertebra 63′ at the distal end of the series where it is anchored. When the curvable portion 70′ is curved, tension builds in the wire rope 66′, which holds the curvable portion 70′ rigid in a left/right torsion position. The second control wire 71B extends sequentially through the each vertebra 61′ in the series at the top of the curvable portion 70′. The second control wire 71B can slide through each vertebra 61′, except for in the first vertebra 63′ where the second control wire 71B is secured. Each of the vertebra 61′ twists on a hinge joint so as to allow the curvable portion 70 to curve in a controlled manner from a fully straight or less curved configuration.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A device to facilitate endotracheal intubation of a patient, comprising:
(a) a support means;
(b) a tubular element, cantilevered at a proximal end from the support means, having an opposed distal end for insertion into the patient's mouth to place an endotracheal tube;
(c) a curvable portion of the tubular element disposed adjacent to the distal end of the tubular element;
(d) a gripping means attached to the support means and disposed forward of the proximal end of the tubular element, such that a length of the tubular element projects over the gripping means;
(e) a control means provided as a component of or adjacent to the gripping means; and
(f) a means for moving the curvable portion transmitting a force applied by the user on the control means to curve the curvable portion, wherein when the control portion is manipulated by the user the curvable portion curves into a generally curved configuration in a controlled manner from a less curved configuration and returns to the less curved conformation when the control means is released.

2. The device of claim 1, wherein the curvable portion comprises a series of interconnected ring elements with spaces therebetween.

3. The device of claim 1, wherein the curvable portion comprises one or more recesses in the tubular element.

4. The device of claim 3, wherein the recesses are provided as slits in the curvable portion.

5. The device of claim 3, wherein the recesses are provided as wedge shaped cuts in the curvable portion.

6. The device of claim 3, wherein the tubular element is constructed of stainless steel or a shape memory alloy (SMA).

7. The device of claim 6, wherein the tubular element is constructed of Nitinol.

8. The device of claim 1, further comprising an insufflation attachment on the tubular element to clear secretions from the patient's airway and supply oxygenation.

9. The device of claim 1, wherein the means for moving the curvable portion is a wire attached to the control means.

10. The device of claim 1, wherein the control means is a trigger mounted on the gripping means.

11. The device of claim 1, further comprising a visualizing means at a proximal end of the device for visualizing of an image of the throat of the patient when the distal end of the tubular element is advanced forward during the endotracheal intubation procedure.

12. A method of inserting an endotracheal tube into the trachea of a patient comprising:
(a) providing a device to facilitate endotracheal intubation of a patient, comprising a support means, a tubular element, cantilevered at a proximal end from the support means, having a distal end for insertion into the patient's mouth to place the endotracheal tube, a curvable portion of the tubular element disposed adjacent to the distal end of the tubular element, a gripping means attached to the support means and disposed forward of the proximal end of the tubular element, such that a length of the tubular element projects over the gripping means, a control means provided as a component of or adjacent to the gripping means, and a means for moving the curvable portion transmitting a force applied by the user on the control means to curve the curvable portion, wherein when the control portion is manipulated by the user the curvable portion curves into a generally curved configuration in a controlled manner from a less curved configuration and returns to the less curved conformation when the control means is released;
(b) sliding the endotracheal tube over the tubular element of the device;
(c) inserting the distal end of the tubular element with the endotracheal tube into the patient's mouth;
(d) manipulating the control means to curve the curvable portion enough so that the distal end of the tubular element can be safely advanced in the throat of the patient;
(e) advancing the distal end of the tubular element to place the endotracheal tube into the trachea of the patient; and
(f) removing the tubular element from the patient's mouth.

13. The method of claim 12, wherein the curvable portion comprises a series of interconnected ring elements with spaces therebetween.

14. The method of claim 12, wherein the curvable portion comprises one or more recesses in the tubular element.

15. The method of claim 14, wherein the recesses are provided as slits in the curvable portion.

16. The method of claim 14, wherein the recesses are provided as wedge shaped cuts in the curvable portion.

17. The method of claim 14, wherein the tubular element is constructed of stainless steel or a shape memory alloy (SMA).

18. The method of claim 17, wherein the tubular element is constructed of Nitinol.

19. The method of claim 12, further comprising the step of clearing secretions from the patient's airway, after inserting the distal end of the tubular element in step (c), by means of an insufflation attachment on the tubular element.

20. The method of claim 12, wherein the control means is a trigger mounted on the gripping means.

21. The method of claim 20, wherein the control means is manipulated by squeezing in step (d) to curve the curvable portion.

22. A method of inserting an endotracheal tube into the trachea of a patient comprising:
(a) providing a device to facilitate endotracheal intubation of a patient, comprising a support means, a tubular element, cantilevered at a proximal end from the support means, having a distal end for insertion into the patient's mouth to place the endotracheal tube, a curvable portion of the tubular element disposed adjacent to the distal end of the tubular element, a gripping means attached to the support means and disposed forward of the proximal end of the tubular element, such that a length of the tubular element projects over the gripping means, a control means provided as a component of or adjacent to the gripping means, a means for moving the curvable portion transmitting a force applied by the user on the control means to curve the curvable portion, and a visualizing means at a proximal end of the device for visualizing of an image of the throat of the patient, wherein when the control portion is manipulated by the user the curvable portion curves into a generally curved configuration in a controlled manner from a less curved configuration and returns to the less curved conformation when the control means is released;
(b) sliding the endotracheal tube over the tubular element of the device;
(c) inserting the distal end of the tubular element with the endotracheal tube into the patient's mouth;
(d) viewing the image of the throat of the patient on the visualizing means;
(e) manipulating the control means to curve the curvable portion enough so that the distal end of the tubular element can be safely advanced in the throat of the patient;
(f) advancing the distal end of the tubular element to place the endotracheal tube into the trachea of the patient; and
(g) removing the tubular element from the patient's mouth.

23. The method of claim 22, wherein the curvable portion comprises a series of interconnected ring elements with spaces therebetween.

24. The method of claim 22, wherein the curvable portion comprises one or more recesses in the tubular element.

25. The method of claim 24, wherein the recesses are provided as slits in the curvable portion.

26. The method of claim 24, wherein the recesses are provided as wedge shaped cuts in the curvable portion.

27. The method of claim 24, wherein the tubular element is constructed of stainless steel or a shape memory alloy (SMA).

28. The method of claim 27, wherein the tubular element is constructed of Nitinol.

29. The method of claim 22, further comprising the step of clearing secretions from the patient's airway, after inserting the distal end of the tubular element in step (c), by means of an insufflation attachment on the tubular element.

30. The method of claim 22, wherein the control means is a trigger mounted on the gripping means.

31. The method of claim 30, wherein the control means is manipulated by squeezing in step (e) to curve the curvable portion.
